(19) United States
(12) Patent Application Publication
(10) Pub. No.: US 2006/0099250 A1
(43) Pub. Date: May 11, 2006

(54) Use of an aqueous solution of citric acid and a water-soluble sugar like lactitol as granulation liquid in the manufacture of tablets

(76) Inventors: Wei Tian, Minety, Wiltshire (GB); John Langridge, Sint Oedenrode (NL)

Correspondence Address: MCANDREWS HELD & MALLOY, LTD, 500 WEST MADISON STREET, SUITE 3400, CHICAGO, IL, 60661, US

(21) Appl. No.: 10/525,712

(22) PCT Filed: Aug. 20, 2003
(86) PCT No.: PCT/GB03/03654

§ 371 Date: Sep. 19, 2005

(30) Foreign Application Priority Data
Aug. 21, 2002 (GB) 0219516.2
Aug. 23, 2002 (GB) 0219744.0

(51) Int. Cl. A61K 31/70; B27N 3/00; A61K 9/20; A61K 31/19

(52) U.S. Cl. 424/464; 514/23; 514/53; 514/574; 264/109

(57) ABSTRACT

The use of an aqueous solution of citric acid and a highly water-soluble sugar as a binder for the granulation of tablet excipients.

The present invention relates to tablet compositions and methods of making tablets. In particular the invention relates to the use of a water-soluble sugar as a lubricant/anti-adherent during the tablet compression process of tablet compositions containing citric acid.

Citric acid (2-hydroxy-1,2,3-propanetricarboxylic acid), in either monohydrate or anhydrous form, is widely used in pharmaceutical formulations and food products as an acidifying agent, an antioxidant, a buffering agent, a chelating agent or a flavour enhancer. Citric acid monohydrate loses water of crystallisation in dry air or when heated to about 40° C. It is slightly deliquescent in moist air. Citric acid is frequently incorporated into effervescent tablets, chewable tablets and fast disintegrating tablets.

Fast disintegrating tablets for oral administration are known. These tablets are readily disintegratable in the mouth, can be taken without water and without chewing.

WO 99/47126 discloses a physiologically acceptable tablet comprising a compressed tablet formulation free of organic solvent residue that rapidly disintegrates when placed in a body cavity, comprising at least one water-soluble non-saccharide polymer, the tablet has a hardness factor of between 0.5 kiloponds and 12.0 kiloponds.

U.S. Pat. No. 5,576,014 discloses intrabucally dissolving compressed mouldings comprising a saccharide having low mouldability having been granulated with a saccharide having high mouldability. The mouldings exhibit quick disintegration and dissolution in the buccal cavity and have an adequate hardness.

U.S. Pat. No. 6,024,981 discloses a hard, compressed, rapidly dissolvable dosage form adapted for direct oral dosing comprising an active ingredient and a matrix including a non-direct compression filler and a lubricant, the dosage form being adapted to rapidly dissolve in the mouth of a patient and thereby liberate the active ingredient.

U.S. Pat. No. 4,886,669 discloses a water-dispersible tablet comprising:
a) microparticles which contain at least one pharmaceutically active substance
b) at least one disintegrant and
c) a swellable material which is able to generate a high viscosity when coming into contact with water and which is selected from guar gum, xanthan gum, alginates, dextran, pectins, polysaccharides, sodium or calcium carboxymethylcellulose, hydroxypropylcellulose and hydroxypropylmethylcellulose,
   which tablet disintegrates rapidly in water forming a homogeneous suspension of high viscosity that can easily be swallowed.

WO99/44580 discloses a formulation for preparing a fast disintegrating tablet comprising a drug in multiparticulate form, one or more water insoluble excipients, one or more disintegrants; and optionally one or more substantially water-soluble excipients, the amount of the ingredients being such as to provide a disintegration time for the tablet in the mouth in the order of seventy five seconds or less. It is stated superior tablet properties can be achieved by choosing appropriate amounts of the ingredients according to the classification shown below:
a) insoluble ingredient this includes the amount of drug either coated or uncoated and the amount of insoluble excipients including the insoluble inorganic salts used as filler diluents (e.g. di- or tri-basic calcium phosphate), organic filler (e.g. microcrystalline cellulose) or water insoluble lubricant (e.g. magnesium stearate, sodium steary fumarate, stearic acid or glyceryl behenate) and glidant (e.g. talc, silicone dioxide etc.).
b) substantially soluble components e.g. the amount of compression sugars (e.g. lactose), flavouring agents, sweeteners, binders and surfactants etc.
c) disintegrant, especially super-disintegrant, such as, maize starch or modified starches, cross-linked polyvinylpyrrolidone or sodium carboxymethylcellulose.

For constant ratios of ingredients a) and b) increasing the amount of disintegrant generally gives poorer friability values and increased disintegration times. In view of this the amount of super disintegrant c) should not be excessive and is therefore preferably in the range 0.5 to 30%, most preferably 1 to 20%, most preferably 2 to 15% by weight of tablet.

British Patent Application No. 0204771.0 discloses a fast disintegrating tablet comprising an active ingredient and one or more disintegrants characterised in that disintegrant or a combination of disintegrants is present in the form of agglomerates having an average agglomerated particle size of at least 50 microns, said agglomerates comprising at least 10% by weight of disintegrant.

EP 0454396 discloses a pharmaceutical tablet composition for active compounds in free base form having one or more undesirable tabletting properties comprising:

a premixture consisting essentially of from about 85 to about 99.9 percent by weight of said premixtures of said active compound and from about 0.1 to about 15 percent of said premixtures of citric acid; and,

one or more additional formulation ingredients.

Effervescence is defined as the evolution of bubble of gas from a liquid, as the result of a chemical reaction. Effervescent mixtures have been known and used medicinally for many years. The effervescent tablets can be either dissolved in water to provide a carbonated or sparkling liquid drink for ingestion or directly placed in the oral cavity where the effervescence facilitates tablet disintegration. Citric acid and sodium bicarbonate are the most commonly used effervescent agents, as disclosed for example, in WO95/23594, WO00/38657 and U.S. Pat. No. 6,071,539.

Chewable tablets are often desired for its convenience and patient acceptance (e.g. for young children and some geriatric patients who can not swallow tablet easily) and for rapid onset of bioactivity (such as obtained from a chewable antacid or anthelmintic tablet) (Darueala, J. B. (1980) Chewable tablets, in Pharmaceutical dosage forms: tablets, Volume I, eds. Lieberman, H. A. and Lachman, L.).

Tablets are made by compressing a granular formulation on a tablet press. The tablet press typically has a set of tooling consisting of a die, an upper punch and a lower punch. Sheth, B. B., Bandelin F. J. and Shangraw R. F. (Compressed tablets, in Pharmaceutical dosage forms: tablets, Volume I, eds. Lieberman, H. A and Lachman, L. (1980)) describe the compression process in several stages: the first stage is the filling cycle during which the lower punch is lowered to a preset point to form a cavity in the die to provide a volume corresponding to the correct fill weight for the tablet. Next the upper punch descends into the die to compress the tablet. Then the lower punch is raised flush with the surface of the die tablet so the tablet can be ejected.

Tablet presses operate at production rates up to a few thousand tablets a minute. Hence, a tablet formulation must first be prepared in a suitable form for compression on a tablet press. This process is referred as granulation. Wet granulation is often the preferred granulation process, which consists of the following basic unit operations:
1. Preparation of powder mixture with screening and mixing
2. Addition of binder solution and mixing with powder to appropriate wetness
3. Drying the solid-liquid blend
4. Milling the dry granulation to size
5. Addition of lubricant, glidant, and/or other excipients prior to compression

Generally it is important that effervescent tablets, or chewable tablets or fast disintegrating tablets have a pleasing taste, flavour and mouthfeel to ensure patient compliance. A key function of citric acid in these formulations is flavour enhancement. To attain a smooth flavour/taste sensation, it is preferable to have citric acid evenly distributed within the tablet matrix. One way of obtaining the even distribution of citric acid is to use it in an aqueous solution as the granulating liquid for the preparation of tablet granules. However, it has proved difficult to prepare tablets from such granules due to extensive sticking during compression.

Sticking refers to the adhesion on the punch faces and occurs when tablets do not leave the punch faces clean. The tablet faces become dull and/or pitted during compression, and the condition progressively worsens to the point where the tablets chip and break and are hard to remove from the lower punch or to pull apart from the upper. Lubricants or anti-adherents are added to the granulation mixture to resolve the problems of sticking.

The primary function of tablet lubricants is to reduce the friction arising at the interface of tablet and die wall during compression and ejection. The primary function of anti-adherents is to prevent sticking to the punch and to a lesser extent, the die wall. With many materials these functions are interchangeable and are difficult to separate completely. Common lubricants and anti-adherents are magnesium stearate, stearic acid, talc, calcium stearate, sodium stearate, sodium lauryl sulfate etc. Magnesium stearate is frequently the preferred lubricant/anti-adherent at an application level 0.25 to 2%. The sticking problem associated with using citric acid solution as granulation liquid cannot be resolved with an increasing amount of conventional lubricants and anti-adherents.

It has been surprisingly found that this problem can be successfully resolved with the incorporation of a water-soluble sugar in the citric acid solution for granulation.

According to one aspect of the invention there is provided the use of an aqueous solution of citric acid and a highly water-soluble sugar as a binder for the granulation of tablet excipients.

According to a second aspect of the invention there is provided a composition for compressing into tablets comprising granules of tablet excipients in which the granules comprise citric acid and highly water-soluble sugar as binder.

According to a further aspect of the invention there is provided a tablet comprising granules of tablet excipient in which said granules comprise citric acid and highly water-soluble sugar as binder.

According to a further aspect of the invention there is provided a method of making a tablet comprising the steps of:

(i) granulating tablet excipients using an aqueous solution of citric acid and a highly water-soluble sugar as a binder,

(ii) drying the granules and optionally reducing the size of the dried granules,

(iii) compressing said dried granules, optionally with additional tablet excipients in a tablet press to form a tablet, wherein the presence of said highly water-soluble sugar acts as a lubricant/anti-adherent in the tablet press.

Any suitable food grade or pharmaceutical grade citric acid can be used in the present invention. The citric acid can be present either in the monohydrate crystalline form or in the anhydrous form.

Highly water-soluble sugars are referred to those substances based on simple crystalline C5 or C6 sugar structures. The sugars can be mono-, di-, tri- and polysaccharides with the degree of polymerisation of less than 20. Preferably the degree of polymerisation is less than 10.

Examples of highly water-soluble sugars are glucose, sucrose, maltose, lactose, arabinose, xylose, ribose, fructose, mannose, galactose, sorbose, trehalose, sorbitol, xylitiol, mannitol, maltitol, lactitol, isomaltol, maltodextrin, hydrogenated starch hydrolysed products. The preferred sugars include maltitol, lactitol, sucrose, trehalose and mixtures thereof.

Preferably, the weight ratio of citric acid to the highly water-soluble sugar used in the aqueous solution is from 1:10 to 10:1; more preferably, 2:10 to 10:2; most preferably, from 5:10 to 10:5.

The citric acid is generally present in an amount of from 1 to 10% by weight based on the granules in which it is present.

The tablet excipients may be selected from a wide range of ingredients known in tablet compositions in the art. The precise selection which will depend upon the desired properties of tablet to be formed e.g. fast disintegrating, sustained release, effervescent, chewable etc. Non-limiting tablet excipients include binders, disintegrants, diluents, active ingredients e.g. drugs, antibiotics etc., flavouring, flow aids, surfactants etc.

The invention will be illustrated by the following Examples in which the following ingredients were used:

| | |
|---|---|
| Mannitol SD200: | mannitol having an average particle size of |
| | about 200 μm manufactured by Roquette |
| Citric acid: | manufactured by Tate & Lyle Ltd |
| Polyplasdone ® XL-10: | crospovidone having an average particle |
| | size of about 30 μm |
| Mannitol: | mannitol having an average particle size of |
| | about 60 μm |
| Explotab ®: | sodium starch glycolate having an average |
| | particle size about 40 μm |

Tablets were prepared using a Mannesty F3 press using normal 10 mm concave toolings. The toolings (upper punch, lower punch and die) were regularly examined for any signs of sticking during compression.

All parts and percentages are by weight unless otherwise stated.

### EXAMPLES

### Example 1 (Comparative)

A fast disintegrating tablet was prepared by wet granulating mannitol (85.3 parts), sodium starch glycolate (Explotab) (5.0 parts) and aspartame (0.5 part) using a citric acid (5.0 part) solution. The wet granules were then dried in a forced air oven at 55° C. to a moisture content of less than 1%. The dried granules were then screened through 1 mm sieve and combined with 4.0 parts of crospovidone (Polyplasdone XL-10), 0.1 part lemon flavour and 0.1 part orange flavour to give a total of 100 parts of granulation A.

Granulation A is then combined with lubricants for tabulating studies.

| | B | C | D | E | F | G | H |
|---|---|---|---|---|---|---|---|
| Granulation A | 95.0 | 92.0 | 94.0 | 93.0 | 95.0 | 97.0 | 89.0 |
| Mg stearate | — | — | 1.0 | — | — | 1.0 | 1.0 |
| Talc | — | 3.0 | — | — | 3.0 | — | 3.0 |
| Na stearyl | — | — | — | 2.0 | 2.0 | 2.0 | 2.0 |
| fumerate | | | | | | | |
| Glyceral | 5.0 | 5.0 | 5.0 | 5.0 | — | — | 5.0 |
| behenate | | | | | | | |
| Total | 100.0 | 100.0 | 100.0 | 100.0 | 100.0 | 100.0 | 100.0 |

There were evidences of sticking with all the lubricant systems used.

### Example 2 (Comparative)

A fast disintegration tablet was prepared according to the formulation described below:

| Formulation component | % w/w |
|---|---|
| Mannitol | 90.7 |
| Citric acid | 2.3 |
| Crospovidone | 6.0 |
| Magnesium stearate | 1.0 |
| Total | 100.0 |

Citric acid was used to granulate mannitol. The granulation process was as described in Example 1. The dried granules were then combined with crospovidone and magnesium stearate for tableting studies.

There was evidence of sticking.

### Example 3 (Invention)

A fast disintegration tablet was prepared according to the formulation described below:

| Formulation component | % w/w |
|---|---|
| Mannitol | 88.4 |
| Citric acid | 2.3 |
| Maltitol | 2.3 |
| Crospovidone | 6.0 |
| Magnesium stearate | 1.0 |
| Total | 100.0 |

Citric acid/maltitol was used to granulate mannitol. The granulation process was as described in Example 1. The dried granules were then combined with crospovidone and magnesium stearate for tabulating studies.

There was no evidence of sticking.

### Example 4 (Invention)

A fast disintegrating tablet was prepared incorporating two separate granule formulations:

| Formulation component | Granulation A | Granulation B |
|---|---|---|
| Mannitol SD200 | 91.0 | — |
| Mannitol M60 | — | 60.0 |
| Explotab | 4.0 | — |
| Citric acid | 2.5 | 7.5 |
| Lactitol | 2.5 | 7.5 |
| Crospovidone | — | 25.0 |
| Total | 100 | 100.0 |

Both granules were granulated with citric acid/lactitol solution. The granulation procedure was as described in Example 1. The tablet formulation used was as follows:

| Formulation component | % w/w |
|---|---|
| Granule A | 74.3 |
| Granule B | 24.0 |
| Aspartame | 0.5 |
| Lemon flavour | 0.2 |
| Magnesium stearate | 1.0 |
| Total | 100.0 |

The tablets were pressed to 1.8 kp, with a mean tablet weight of 248.7 mg, a mean thickness of 4.2 mm and a diameter of 10.1 mm. These tablets had a friability of 0.63% according to the standard USP friability method and an oral disintegration time of 15 seconds.

There was no evidence of sticking.

### Example 5 (Invention)

Formulation of Sildenafil Tablet

| Formulation component | mg/tablet |
|---|---|
| Sildenafil granules | 185.75 |
| Agglomerated disintegrant granules | 60.00 |
| Aspartame | 1.25 |
| Lemon flavour | 0.50 |
| Magnesium stearate | 2.50 |
| Total | 250.0 |

Sildenafil Granules were Prepared According to the Following Formulation:

| Formulation component | % |
|---|---|
| Sildenafil citrate | 37.81 |
| Mannitol SD200 | 45.19 |
| Vivastar (sodium starch glycolate) | 7.00 |
| Citric acid | 5.00 |
| Lactitol | 5.00 |
| Total | 100.00 |

To prepare the sildenafil granule, citric acid and lactitol were dissolved in water. Sildenafil citrate, mannitol SD200 and sodium starch glycolate were blended in a food processor for 10 minutes, the citric acid/lactitol solution was added and mixed in, and the resulting wet granules were dried in a tray drier at 50° C.

Agglomerated disintegrant granules were prepared according to the following formulation:

| Formulation component | % |
|---|---|
| Mannitol (M60) | 60.00 |
| Polyplasdone XL-10 | 25.00 |
| Citric acid | 7.50 |
| Lactitol | 7.50 |
| Total | 100.00 |

To prepare the agglomerated disintegrant granules, citric acid and lactitol were dissolved in deionised water, mannitol and polyplasdone were dry mixed for 10 minutes in a food mixer. The citric acid/lactitol solution was added to the dry mixture to form wet granules. The wet granules were dried in a forced air oven at 50° C. to achieve a moisture level of less than 2%. The dried granules were screened and the 75 to 250 micron size range was obtained.

Tableting: the sildenafil granules and agglomerated disintegrant granules were placed in a suitable container. Aspartame and lemon flavour were screened, added to the mixture and blended for 10 minutes. Magnesium stearate was screened, added to the mixture and blended for a further 2 minutes. Tablets were prepared using a Stoke B2 rotary press fitted with 16 stations of ⅜ inch (9.525 mm) normal concave tooling. There was no evidence of sticking.

The tablets had an average weight of 252 mg and a mean crushing strength of 1.1 kp. The oral disintegration time was 28 seconds.

### Example 6 (Invention)

Tablets Incorporating Concentrated Sildenafil Granules

Formulation of Sildenafil Tablet:

| Formulation component | mg/tablet |
|---|---|
| Sildenafil granules | 76.77 |
| Mannitol granules | 107.73 |
| Agglomerated disintegrant granules | 60.00 |
| Aspartame | 2.00 |
| Lemon flavour | 1.00 |
| Magnesium stearate | 2.50 |
| Total | 250.0 |

Sildenafil Granules were Prepared According to the Following Formulation:

| Formulation component | % |
|---|---|
| Sildenafil citrate | 91.50 |
| Lemon flavour | 1.00 |
| Aspartame | 2.50 |
| Citric acid | 2.50 |
| Lactitol | 2.50 |
| Total | 100.00 |

To prepare the slidenafil granule, citric acid and lactitol were dissolved in water. Sildenafil citrate, lemon flavour and aspartame were blended in a food processor for 10 minutes, the citric acid/lactitol solution was added and mixed in, and the resulting wet granules were dried in a tray drier at 50° C.

Mannitol granules were prepared according to the following formulation.

| Formulation component | % |
|---|---|
| Mannitol (SD200) | 91.00 |
| Vivastar (sodium starch glycolate) | 4.00 |
| Citric acid | 2.50 |
| Lactitol | 2.50 |
| Total | 100.00 |

To prepare the mannitol granules, citric acid and lactitol were dissolved in deionised water, mannitol and Vivastar were mixed for 10 minutes in a food mixer. The citric acid/lactitol solution was added to the dry mixture to form wet granules. The wet granules were dried in a forced air oven at 50° C. to achieve a moisture level of less than 1%.

Agglomerated disintegrant granules were prepared according to Example 5.

Tableting: the sildenafil granules, mannitol granules, agglomerated disintegrant granules were placed in a suitable container. Aspartame and lemon flavour were screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed). There was no evidence of sticking.

The tablets had an average weight of 252.5 mg and a mean crushing strength of 1.1 kp. The oral disintegration time was 12 seconds demonstrating the significant improvement in oral disintegration time when concentrated sildenafil granules were incorporated.

### Example 7 (Invention)

Tablets Incorporating Concentrated Sildenafil Granules and an Increased Amount of Sweetener

Formulation of Sildenafil Tablet:

| Formulation component | mg/tablet |
|---|---|
| Sildenafil granules | 90.00 |
| Mannitol granules | 95.00 |
| Agglomerated disintegrant granules | 60.00 |
| Lemon flavour | 2.50 |
| Magnesium stearate | 2.50 |
| Total | 250.0 |

Sildenafil granules were prepared according to the following formulation:

| Formulation component | % |
|---|---|
| Sildenafil citrate | 78.04 |
| Acesulfame K (high intensity | 16.40 |
| sweetener) | |
| Citric acid | 2.78 |
| Lactitol | 2.78 |
| Total | 100.00 |

To prepare the sildenafil granules, citric acid and lactitol were dissolved in water. Sildenafil citrate and acesulfame K were blended in a food processor for 10 minutes, the citric acid/lactitol solution was added and mixed in, and the resulting wet granules were dried in a tray drier at 50° C.

Mannitol granules were prepared according to Example 6.

Agglomerated disintegrant granules were prepared according to Example 5.

Tableting: the slidenafil granules, mannitol granules, agglomerated disintegrant granules were placed in a suitable container. Lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed). There was no evidence of sticking.

The tablets had an average weight of 251.1 mg and a mean crushing strength of 1.4 kp. The oral disintegration time was 15 seconds demonstrating the significant improvement in oral disintegration time when concentrated sildenafil granules were incorporated. The tablets had a strong bitter taste which lingered in the mouth for more than 5 minutes suggesting that the bitter taste can not be successfully masked by sweetener alone.

### Example 8 (Invention)

Tablets Incorporating Concentrated Sildenafil Granules and a Solubilisation Inhibitor

Formulation of Sildenafil Tablet:

| Formulation component | mg/tablet |
|---|---|
| Sildenafil granules | 110.20 |
| Mannitol granules | 62.50 |
| Agglomerated disintegrant granules | 60.00 |
| Lemon flavour | 5.00 |
| Acesulfame K | 9.80 |
| Magnesium stearate | 2.50 |
| Total | 250.0 |

Sildenafll granules were prepared according to the following formulation:

| Formulation component | % |
|---|---|
| Sildenafil citrate | 60.50 |
| Acesulfame K | 8.30 |
| Sodium carbonate | 26.20 |
| Citric acid | 2.50 |
| Lactitol | 2.50 |
| Total | 100.00 |

To prepare the sildenafil granules, citric acid and lactitol were dissolved in distilled water. Sildenafil citrate, sodium carbonate and acesulfame K were blended in a food processor for 10 minutes, the citric acid/lactitol solution was added and mixed in, and the resulting wet granules were dried in a tray drier at 50° C.

Mannitol granules were prepared according to Example 6.

Agglomerated disintegrant granules were prepared according to Example 5.

Tableting: the sildenafil granules, mannitol granules, agglomerated disintegrant granules were placed in a suitable container. Acesulfame K and lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed).

There was no evidence of sticking.

The tablets had a pleasant sweet taste without the characteristic bitterness of sildenafil demonstrating the taste masking function of sodium carbonate. It was of interest to note that no effervescence was detected within the mouth.

### Example 9 (Invention)

Tablets Incorporating Concentrated Sildenafil Granules and a Solubilisation Inhibitor.

Formulation of Sildenafil Tablet

| Formulation component | mg/tablet |
|---|---|
| Sildenafil granules | 116.00 |
| Mannitol granules | 58.50 |
| Agglomerated disintegrant granules | 60.00 |
| Lemon flavour | 5.00 |
| Acesulfame K | 8.00 |
| Magnesium stearate | 2.50 |
| Total | 250.0 |

Slidenafil granules were prepared according to the following formulation:

| Formulation component | % |
|---|---|
| Sildenafil citrate | 63.70 |
| Acesulfame K | 8.71 |
| Sodium carbonate | 27.59 |
| Total | 100.00 |

To prepare the sildenafil granule, sildenafil citrate, sodium carbonate and acesulfame K were blended in a food processor for 10 minutes, distilled water was added was added and mixed in, and the resulting wet granules were dried in a tray drier at 50° C.

Mannitol granules were prepared according to Example 6.

Agglomerated disintegrant granules were prepared according to Example 5.

Tableting: the sildenafil granules, mannitol granules, agglomerated disintegrant granules were placed in a suitable container. Acesulfame K and lemon flavour was screened, added to the mixture and blended for 10 minutes. Magnesium stearate was screened, added to the mixture and blended for a further 2 minutes. Tablets were prepared using a Colton 204 rotary press fitted with 4 stations of 10 mm normal concave tooling (chromed). There was no evidence of sticking.

The tablets had an average weight of 260.0 mg and a mean hardness of 0.9 kp. The oral disintegration time was 10 seconds demonstrating the significant improvement in oral disintegration time when concentrated slidenafil granules were incorporated. The tablets had a pleasant sweet taste without the characteristic bitterness of sildenafil demonstrating the taste masking function of sodium carbonate. No effervescence was detected within the oral cavity.

1. A method for the granulation of tablet excipients comprising using an aqueous solution of citric acid and a highly water-soluble sugar as a binder for the granulation of tablet excipients.

2. The method as claimed in claim 1 in which the highly water-soluble sugar is based on simple crystalline C5 or C6 sugar structures and is a mono-, di, tri or polysaccharide with a degree of polymerization of less than 20.

3. The method as claimed in claim 2 in which the highly water-soluble sugar is selected from glucose, sucrose, maltose, lactose, arabinose, xylose, ribose, fructose, mannose, galactose, sorbose, trehalose, sorbitol, xylitiol, mannitol, maltitol, lactitol, isomaltol, maltodextrin, hydrogenated starch hydrolysed products and mixtures thereof.

4. The method as claimed in claim 3 in which the sugar is selected from maltitol, lactitol, sucrose, trehalose and mixtures thereof.

5. The method as claimed in claim 1 in which the weight ratio of citric acid to highly water-soluble sugar is from 1:10 to 10:1.

6. The method as claimed in claim 5 in which the weight ratio of citric acid to highly water-soluble sugar is from 2:10 to 10:2.

7. The method as claimed in claim 1 in which the citric acid is present in an amount of from 1 to 10% by weight based on the granules.

8. A composition for compressing into tablets comprising granules of tablet excipients in which the granules comprise citric acid and highly water-soluble sugar as binder.

9. A composition as claimed in claim 8 in which the highly water-soluble sugar is based on simple crystalline C5 or C6 sugar structures and is a mono-, di, tri or polysaccharide with a degree of polymerization of less than 20.

10. A composition as claimed in claim 9 in which the highly water-soluble sugar is selected from glucose, sucrose, maltose, lactose, arabinose, xylose, ribose, fructose, mannose, galactose, sorbose, trehalose, sorbitol, xylitiol, mannitol, maltitol, lactitol, isomaltol, maltodextrin, hydrogenated starch hydrolysed products and mixtures thereof.

11. The composition as claimed in claim 10 in which the sugar is selected from maltitol, lactitol, sucrose, trehalose and mixtures thereof.

12. A composition as claimed in claim 8 in which the weight ratio of citric acid to highly water-soluble sugar is from 1:10 to 10:1.

13. The composition as claimed in claim 12 in which the weight ratio of citric acid to highly water-soluble sugar is from 2:10 to 10:2.

14. A composition as claimed in any claim 8 in which the citric acid is present in an amount of from 1 to 10% by weight based on the granules.

15. A tablet comprising granules of tablet excipient in which said granules comprise citric acid and highly water-soluble sugar as binder.

16. The tablet as claimed in claim 15 in which the highly water-soluble sugar is based on simple crystalline C5 or C6 sugar structures and is a mono-, di, tri or polysaccharide with a degree of polymerization of less than 20.

17. A tablet as claimed in claim 16 in which the highly water-soluble sugar is selected from glucose, sucrose, maltose, lactose, arabinose, xylose, ribose, fructose, mannose, galactose, sorbose, trehalose, sorbitol, xylitiol, mannitol, maltitol, lactitol, isomaltol, maltodextrin, hydrogenated starch hydrolysed products and mixtures thereof.

18. A tablet as claimed in claim 17 in which the sugar is selected from maltitol, lactitol, sucrose, trehalose and mixtures thereof.

19. A tablet as claimed in claim 15 in which the weight ratio of citric acid to highly water-soluble sugar is from 1:10 to 10:1.

20. A tablet as claimed in claim 19 in which the weight ratio of citric acid to highly water-soluble sugar is from 2:10 to 10:2.

21. A tablet as claimed in claim 15 in which the citric acid is present in an amount of from 1 to 10% by weight based on the granules.

22. A method of making a tablet comprising the steps of:
(i) granulating tablet excipients using an aqueous solution of citric acid and a highly water-soluble sugar as a binder,
(ii) drying the granules and optionally reducing the size of the dried granules,
(iii) compressing said dried granules, optionally with additional tablet excipients in a tablet press to form a tablet, wherein the presence of said highly water-soluble sugar acts as a lubricant/anti-adherent in the tablet press.

23. A method of making a tablet as claimed in claim 22 in which the highly water-soluble sugar is based on simple crystalline C5 or C6 sugar structures and is a mono-, di, tri or polysaccharide with a degree of polymerization of less than 20, preferably less than 10.

24. A method of making a tablet as claimed in claim 23 in which the highly water-soluble sugar is selected from glucose, sucrose, maltose, lactose, arabinose, xylose, ribose, fructose, mannose, galactose, sorbose, trehalose, sorbitol, xylitiol, mannitol, maltitol, lactitol, isomaltol, maltodextrin, hydrogenated starch hydrolysed products and mixtures thereof.

25. A method of making a tablet as claimed in claim 24 in which the sugar is selected from maltitol, lactitol, sucrose, trehalose and mixtures thereof.

26. A method of making a tablet as claimed in claim 22 in which the weight ratio of citric acid to highly water-soluble sugar is from 1:10 to 10:1.

27. A method of making a tablet as claimed in claim 26 in which the weight ratio of citric acid to highly water-soluble sugar is from 2:10 to 10:2.

28. A method of making a tablet as claimed claim 22 in which the citric acid is present in an amount of from 1 to 10% by weight based on the granules.
